(19) United States
(12) Patent Application Publication
(10) Pub. No.: US 2013/0323291 A1
(43) Pub. Date: Dec. 5, 2013

(54) HYDROPHILIC AND NON-THROMBOGENIC POLYMER FOR COATING OF MEDICAL DEVICES

(71) Applicants: Junwei Li, Collegeville, PA (US); William J. Work, Huntingdon Valley, PA (US)

(72) Inventors: Junwei Li, Collegeville, PA (US); William J. Work, Huntingdon Valley, PA (US)

(73) Assignee: BIOCOAT INCORPORATED, Horsham, PA (US)

(21) Appl. No.: 13/484,897

(22) Filed: May 31, 2012

(51) Int. Cl. C09D 133/26; C08K 5/09; B32B 27/08; A61P 31/00; B32B 27/28; C09D 139/06; A61K 9/00

(52) U.S. Cl. 424/409; 524/548; 524/555; 524/300; 522/167; 522/175; 523/122; 524/502; 424/400; 428/500; 428/520; 428/424.2

(57) ABSTRACT

A hydrophilic copolymer is designed and synthesized by copolymerization of an acidic monomer and a second hydrophilic monomer. The copolymer is non-thrombogenic, hydrophilic and incorporates reactive functional groups. The copolymer can then be covalently attached to a primer/base coat through its functional groups, to form a durable lubricious coating on medical devices. A coating formed of the polymer on a surface is non-thrombogenic and non-cytotoxic. The coating shows good stability in gamma ray, e-beam and ethylene oxide sterilization.

### BACKGROUND OF THE INVENTION

The present invention relates to the field of non-thrombogenic and lubricious coatings that are applied to medical devices, especially devices intended to be implanted, temporarily or permanently, in the body and in blood-contact applications.

Among the many advances in medical practice in recent years is the development of medical devices that supplement the surgeon's skills. Examples of these are a variety of vascular catheters and guide wires that can be used to treat remote areas of the circulatory system otherwise available only by major surgery. Another is the stent, a device that reinforces arterial walls and prevents occlusion after angioplasty. Another is the intra-ocular lens that restores youthful eyesight to the elderly afflicted with cataracts. Heart valves, artificial pacemakers, and orthopedic implants are among a lengthening list of other such devices.

Nearly all of the above-described devices are constructed of plastics and metals that were never intended to invade and sometimes reside for prolonged periods in the human body. They present surfaces that bear little or no resemblance to those of human organs, which are generally hydrophilic, slippery and biocompatible.

Equally important for devices that must be inserted and moved through body tissues is their lubricity. Most metals and plastics have poor lubricity against body tissues, which results in mechanical abrasion and discomfort when the device is passed over the tissue.

The surfaces of devices designed and manufactured from such materials can be made biocompatible, as well as hydrophilic and slippery, by properly designed coatings. Thus, the way has been opened to construct medical devices from conventional plastics and metals having the particular physical properties required, and then to apply suitable coatings to impart the desired properties to their surfaces.

It has been shown that polymers that have low coefficients of friction when wet are water soluble polymers that are cross-linked or otherwise immobilized and swell, but do not dissolve, upon exposure to water. Polysaccharides have been shown to be useful in making hydrophilic, lubricious coatings on substrates. Such coatings are described in U.S. Pat. Nos. 4,801,475, 5,023,114, 5,037,677, and 6,673,453, the disclosures of which are hereby incorporated by reference. Lubricious coatings based upon polysaccharides exhibit exceptional biocompatibility and lubricity, but relatively poor resistance to ionizing radiation.

It is desirable for some applications to have a lubricious coating made of a synthetic polymer for the benefits of a longer shelf-life and stability to radiation-sterilization processes. Hydrophilic synthetic polymers, such as poly(acrylic acid) and its copolymers have often been proposed to make lubricious, hydrophilic coatings because of their ability to generate a hydrated layer on the surface.

Many attempts have been made to immobilize poly(acrylic acid) on surfaces so that they may be utilized as coatings on medical devices. The methods in U.S. Pat. Nos. 4,642,267 and 4,990,357 include physical blends of poly(acrylic acid) copolymer with a polyurethane dispersion. This method has the drawback that the interpolymer network physically attaching the hydrophilic polymer to the substrate surface often breaks down upon prolonged turbulent flow or soaking and the hydrophilic species may be washed away thereby rendering the article insufficiently lubricious.

Other methods invented to utilize poly(acrylic acid) as a hydrophilic coating on a surface include radiation grafting of a carboxylic acid monomer and its polymer as described in U.S. Pat. Nos. 2,999,056, 5,531,715, 5,789,018, and 6,221,061, and EP 0669837, plasma grafting of an acrylic acid monomer in EP 0220919, and also methods using a primer layer containing isocyanate, aziridine, amine and hydroxyl functional groups to anchor polyacrylic acid as stated in U.S. Pat. Nos. 5,091,205, 5,136,616, 5,509,899, 5,702,754, 6,048,620, 6,558,798, 6,709,706, 6,087,416, 6,534,559, and EP 0379156, EP 0480809, EP 0728487, and EP 0963761. The disclosures of all of the above-mentioned patents are hereby incorporated by reference.

The above mentioned poly(acrylic acid) coatings exhibit relatively poor lubricity and/or durability because of insufficient hydrophilic polymer coating thickness and/or poor binding to the surface. It is difficult to achieve a high density surface coverage by either grafting through photo-initiated polymerization or surface chemical attachment of polymers. Multiple-repeated coating processes may increase the thickness of photo-initiated polymerization coating, but will greatly decrease productivity and add to the cost of manufacture.

Using a cross-linker can increase the thickness of a hydrophilic coating considerably. The prior art includes methods to cross-link polyacrylic acid coatings by photo radiation and by the reaction of polyfunctional reactive compounds, such as melamine and aziridines, as described in U.S. Pat. Nos. 5,531,715, 6,558,798, and EP 533821. However, the cross-linked hydrophilic coatings in the art often face a trade-off between lubricity and abrasion resistance, which are both indispensable properties for a hydrophilic coating. A highly cross-linked coating has poor lubricity because of its low capacity for hydration and reduced mobility of polymer segments in aqueous media. A coating with a low cross-linking density has a high swelling ratio, which generally leads to poor abrasion resistance and weak mechanical strength.

U.S. Patent Application Pub. No. 2011/0200828 teaches a bilaminar coating that includes a base-coat that firmly adheres to the substrate and a top-coat that is chemically grafted to the base-coat. The top-coat comprises a mixture of a water-soluble polymer containing carboxylic acid groups and a water soluble chromium (III) compound. The coating forms a very durable, lubricious layer when wet. However, the carboxylate anion comprising the coating shows poor performance in thrombogenicity tests, such as the partial thromboplastin time (PTT) test. The disclosure of the above-cited reference is hereby incorporated by reference.

Contacting blood with a foreign object having a plastic or metal surface induces a complex set of clot-forming reactions that occur at the blood surface interface. Thromboembolism is a major complication associated with the clinical use of artificial devices, such as catheters, guidewires, mechanical heart valves, ventricular assist devices, implantable artificial hearts, vascular grafts, etc. In particular, thromboembolism is an important complication of angiographic procedures, particularly with catheter and guidewire manipulations proximal to the brachiocephalic vessels.

Surface modification is commonly used to make the materials more blood-compatible, while minimizing any loss of mechanical properties. Two approaches to modification have been commonly used. Suppression of nonspecific protein adsorption using coatings of polyethylene oxide (PEO) (a neutral, hydrophilic, and highly flexible polymer) or other hydrophilic polymers has been investigated for surface passivation. Uncontrolled, nonspecific protein adsorption, which usually occurs within seconds following the exposure of a foreign surface to blood, can initiate blood coagulation and the complement pathways.

A second approach has been to use coatings that actively assist the anticoagulant activity of surfaces. Certain plasma proteins (such as antithrombin (AT) which can inhibit thrombin and factor Xa (FXa)) or heparin (a glycosaminoglycan which catalyzes the reactions of plasma AT) have been used for this purpose. Frech et al., in “A Simple Noninvasive Technique to Test Nonthrombogenic Surfaces,” The American Journal of Roentgenology, vol. 113 (1971), p. 765-768, discloses coating of a guidewire with a benzalkonium-heparin complex. Ovitt et al., in “Guidewire Thrombogenicity and Its Reduction”, Radiology, vol. 111 (1974), p. 43-46, reports Teflon coated guidewires treated with benzalkonium-heparin. U.S. Pat. No. 4,349,467 (William) shows the application of heparin to solid polymeric resin substrates by steeping the substrate in a solution of an ammonium salt and contacting the substrate with a heparin salt solution.

There have also been many attempts to invent hydrophilic polymers with applications ranging from electrophoresis, hair treatment and paper treatment. As revealed by Albarghouthi et al, in “Poly-N-hydroxyethylacrylamide (polyDuramide): A novel, hydrophilic, self-coating polymer matrix for DNA sequencing by capillary electrophoresis”, Electrophoresis, vol. 23 (2002), p. 1429-1440, non-ionic monomers, such as N-hydroxyethyl acrylamide, have great hydrophilicity.

The following references, namely WO10041527A, WO10041530A, WO11125713A, and WO09122845A, JP2011046619A, JP2011046652A, JP2010126482A, JP2010090049A, teach copolymers comprised of a 5-30 mol % of a carboxylic acid monomer and 70-95 mol % of an alcohol containing acrylic monomer for use in hair treatment formulations. These patent applications do not disclose the utility of the copolymers as lubricious, biocompatible coatings nor do they disclose their resistance to ionizing radiation. JP2006176934A teaches copolymers from methacrylamide, hydroxyethyl acrylamide, and an ionic vinyl monomer for use as an additive to increase the strength of the paper. The latter reference does not disclose the utility of the copolymers as lubricious, biocompatible coatings nor does it disclose their resistance to ionizing radiation.

### SUMMARY OF THE INVENTION

The present invention comprises a method for rendering a surface of a preformed article to be both lubricious and non-thrombogenic. Hydrophilic and non-thrombogenic polymers are formed by proper design and polymerization. The hydrophilic polymers are then chemically grafted to a base-coat that is firmly adhered to a substrate. The bilaminar coating has good performance in thrombogenicity tests, at the same time the coating imparts excellent lubricity and durability when wet, revealed by pinch testing. The coating also possesses good stability in gamma and E-beam sterilizations. In this invention, the blood compatibility of the ionic polymer coating is greatly improved by incorporation of non-ionic and hydroxyl group containing monomers in the polymers.

In the present invention, a hydrophilic copolymer is designed and synthesized by copolymerization of an acidic monomer and a second non-ionic hydrophilic monomer. This invention teaches a polymer composition that improves the blood compatibility of anionic polymers by incorporation of non-ionic hydrophilic monomers. The copolymer is non-thrombogenic, hydrophilic and incorporates reactive functional groups.

The copolymer of the present invention can be covalently attached to a primer/base coat through its functional groups, to form a durable lubricious coating on medical devices. A coating formed of the polymer on a surface is non-thrombogenic and non-cytotoxic. The coating shows good stability in gamma ray, e-beam and ethylene oxide sterilization.

The present invention also comprises a substrate, typically a device intended to be implanted temporarily or permanently in the human body, having a bilaminar coating. The bilaminar coating includes a base-coat that firmly adheres to the substrate and a top-coat that is chemically grafted to the base-coat and cross-linked. The top-coat forms a non-thrombogenic, hydrophilic, lubricious layer on the surface of the substrate.

In the present invention, the top-coat comprises a water-soluble polymer containing carboxylic acid groups, hydroxyl groups, and other hydrophilic functional groups, which forms a coating with a three dimensional network structure when it is cured.

Another aspect of the invention is that the crosslinking reaction proceeds slowly, if at all, in the aqueous mixture of the hydrophilic polymer. It is only during the drying and curing processes that the top-coat polymer is crosslinked.

The hydrophilic top-coat is grafted to a highly adherent base-coat. The mechanical strength of the hydrated coating is greatly increased through covalent bonding to the base-coat, while its lubricity is retained. The coated products display a combination of adhesion, abrasion resistance, water resistance, gamma-sterilization stability, biocompatibility, and lubricity.

The base-coat and top-coat also contain functional groups that enable the two coats to be chemically grafted to each other. Preferably, the base-coat polymer contains multifunctional isocyanate and multifunctional aziridine groups that react with the top-coat polymer and form chemical bonds between the top-coat and the base-coat.

The present invention therefore has the primary objective of providing a lubricious, biocompatible coating for a medical device.

The invention has the further object of providing a coating as described above, wherein the coating is non-thrombogenic.

The invention has the further objective of providing a coating as described above, wherein the coating can be gamma-ray sterilized.

The invention has the further objective of providing a coating as described above, wherein the coating can be e-beam sterilized.

The invention has the further objective of providing a coating as described above, wherein the hydrated coating is highly durable, resistant to water and salt solutions such as PBS and abrasion resistant.

The reader skilled in the art will recognize other objects and advantages of the present invention from a reading of the following brief description of the drawings, the detailed description of the invention, and the appended claims.

### BRIEF DESCRIPTION OF THE DRAWINGS

FIG. 1 provides a graph showing the coefficient of friction of coatings prepared, according to the present invention, from the hydrophilic copolymers P(HEAA-AA) 50/50 by mol, P(HEAA-AA) 75/25 by mol, P(HEAA-AA) 95/5 by mol, and P(NVP-AA) 50/50 by mol, respectively. The coatings have a bi-laminar structure with said hydrophilic copolymer as the top-coat and an acrylate coating, HYDAK B-500, as the base-coat. The coatings are applied on copolyester rods. Frictional properties of the samples are tested by using a pinch tester in 37° C. phosphate buffer solution. The frictional force is recorded by a load cell and the frictional coefficient is calculated by dividing frictional force with the applied pinch force.

FIG. 2 provides a graph showing the durability of the coatings made, according to the present invention, from hydrophilic copolymers: P(HEAA-AA) 50/50 by mol, P(HEAA-AA) 75/25 by mol, P(HEAA-AA) 95/5 by mol, and P(NVP-AA) 50/50 by mol, respectively. The coatings have a bi-laminar structure with said hydrophilic copolymer as the top-coat and an acrylate coating, HYDAK B-500, as the base-coat. The coatings are applied on copolyester rods. The samples are tested by using a pinch tester and the durability of the coatings is measured by the total friction growth (%) of 100 cycles in the pinch testing. Smaller growth of frictional force with testing cycle indicates better durability.

FIG. 3 provides a graph showing the coefficient of friction of coatings prepared according to the present invention, after gamma sterilization and accelerated aging. The coating is prepared by using P(HEAA-AA) 75/25 by mol as the top-coat and an acrylate coating, HYDAK B-500, as the base-coat. The coating is applied on copolyester rods. Three lots of coated rods, JL 100511, JL100611 and JL101211 are prepared by using coatings with same composition but different batches of raw materials. The coated samples are sterilized by gamma-ray radiation. The sterilized samples are subjected to accelerated aging at 52° C. The samples are tested by using a pinch tester in 37° C. phosphate buffer solution. The frictional force is recorded by a load cell and the frictional coefficient is calculated by dividing frictional force with the applied pinch force. In the chart the lighter columns are the results of gamma sterilized samples without aging, while the darker columns are the results of sterilized samples after 45 days aging at 52° C.

FIG. 4 provides a graph showing the durability of the coatings made according to the present invention, after gamma sterilization and accelerated aging. The coating is prepared by using P(HEAA-AA) 75/25 by mol as the top-coat and an acrylate coating, HYDAK B-500, as the base-coat. The coating is applied on copolyester rods. Three lots of coated rods, JL100511, JL100611 and JL 101211 are prepared by using coatings with same composition but different batches of raw materials. The coated samples are sterilized by gamma-ray radiation. The sterilized samples are subjected to accelerated aging at 52° C. The samples are tested by using a pinch tester in 37° C. phosphate buffer solution. The durability of the coatings is measured by the total friction growth (%) of 50 cycles in the pinch testing. Smaller growth of frictional force with testing cycle indicates better durability. In the chart the lighter columns are the results of gamma sterilized samples without aging, while the darker columns are the results of sterilized samples after 45 days aging at 52° C.

FIG. 5 provides a graph showing the coefficient of friction of coatings made according to the present invention, after E-beam sterilization and accelerated aging. The coating is prepared by using P(HEAA-AA) 75/25 by mol as the top-coat and an acrylate coating, HYDAK B-500, as the base-coat. The coating is applied on copolyester rods. Three lots of coated rods, JL100511, JL100611 and JL101211 are prepared by using coatings with same composition but different batches of raw materials. The coated samples are sterilized by E-beam radiation. The sterilized samples are subjected to accelerated aging at 52° C. The samples are tested by using a pinch tester in 37° C. phosphate buffer solution. The frictional force is recorded by a load cell and the frictional coefficient is calculated by dividing frictional force with the applied pinch force. In the chart the lighter columns are the results of E-beam sterilized samples without aging, while the darker columns are the results of sterilized samples after 45 days aging at 52° C.

FIG. 6 provides a graph showing the durability of the coatings made according to the present invention after E-beam sterilization and accelerated aging. The coating is prepared by using P(HEAA-AA) 75/25 by mol as the top-coat and an acrylate coating, HYDAK B-500, as the base-coat. The coating is applied on copolyester rods. Three lots of coated rods, JL100511, JL100611 and JL101211 are prepared by using coatings with same composition but different batches of raw materials. The coated samples are sterilized by E-beam radiation. The sterilized samples are subjected to accelerated aging at 52° C. The samples are tested by using a pinch tester in 37° C. phosphate buffer solution. The durability of the coatings is measured by the total friction growth (%) of 50 cycles in the pinch testing. Smaller growth of frictional force with testing cycle indicates better durability. In the chart the lighter columns are the results of E-beam sterilized samples without aging, while the darker columns are the results of sterilized samples after 45 days aging at 52° C.

FIG. 7 provides a graph showing the coefficient of friction of coatings made according to the present invention, after ethylene oxide sterilization and accelerated aging. The coating is prepared by using P(HEAA-AA) 75/25 by mol as the top-coat and an acrylate coating, HYDAK B-500, as the base-coat. The coating is applied on copolyester rods. Three lots of coated rods, JL 100511, JL100611 and JL101211 are prepared by using coatings with same composition but different batches of raw materials. The coated samples are sterilized by ethylene oxide gas. The sterilized samples are subjected to accelerated aging at 52° C. The samples are tested by using a pinch tester in 37° C. phosphate buffer solution. The frictional force is recorded by a load cell and the frictional coefficient is calculated by dividing frictional force with the applied pinch force. In the chart the lighter columns are the results of ethylene oxide sterilized samples without aging, while the darker columns are the results of sterilized samples after 45 days aging at 52° C.

FIG. 8 provides a graph showing the durability of the coatings made according to the present invention, after ethylene oxide sterilization and accelerated aging. The coating is prepared by using P(HEAA-AA) 75/25 by mol as the top-coat and an acrylate coating, HYDAK B-500, as the base-coat. The coating is applied on copolyester rods. Three lots of coated rods, JL100511, JL100611 and JL101211 are prepared by using coatings with same composition but different batches of raw materials. The coated samples are sterilized by ethylene oxide gas. The sterilized samples are subjected to accelerated aging at 52° C. The samples are tested by using a pinch tester in 37° C. phosphate buffer solution. The durability of the coatings is measured by the total friction growth (%) of 50 cycles in the pinch testing. Smaller growth of frictional force with testing cycle indicates better durability. In the chart the lighter columns are the results of ethylene oxide sterilized samples without aging, while the darker columns are the results of sterilized samples after 45 days aging at 52° C.

FIG. 9 provides a graph showing the coefficient of friction of coatings made according to the present invention, after prolonged soaking in 37° C. phosphate buffer solution (PBS). The coating is prepared by using P(HEAA-AA) 75/25 by mol as the top-coat and an acrylate coating, HYDAK B-500, as the base-coat. The coating is applied on copolyester rods. Two lots of coated rods, JL112111X and JL112111Y are prepared by using coatings with same composition but different batches of raw materials. The samples are soaked in 37° C. PBS for up to 16 hours before being tested using a pinch tester in 37° C. PBS. The frictional force is recorded by a load cell and the frictional coefficient is calculated by dividing frictional force with the applied pinch force. In the chart the light, darker, and darkest columns are the results of samples soaked in 37° C. of PBS for 0, 7 and 16 hours, respectively, before testing.

FIG. 10 provides a graph showing the durability of the coatings made according to the present invention, after prolonged soaking in 37° C. phosphate buffer solution (PBS). The coating is prepared by using P(HEAA-AA) 75/25 by mol as the top-coat and an acrylate coating, HYDAK B-500, as the base-coat. The coating is applied on copolyester rods. Two lots of coated rods, JL112111X and JL112111Y are prepared by using coatings with same composition but different batches of raw materials. The samples are soaked in 37° C. PBS for up to 16 hours before being tested by using a pinch tester in 37° C. PBS. The samples are tested by using a pinch tester and the durability of the coatings are measured by total friction growth (%) of 50 cycles in the pinch testing. Smaller growth of frictional force with testing cycle indicates better durability. In the chart the light, darker, and darkest columns are the results of samples soaked in 37° C. of PBS for 0, 7 and 16 hours, respectively, before testing.

### DETAILED DESCRIPTION OF THE INVENTION

The requirements for any coating intended for use on medical devices will be set forth and explained first. The specification will then show how the present invention fulfills these requirements.

The coating of the present invention must have the following properties:

(1) It must be able, on drying, to form a continuous, adherent film of good integrity on the surface of the material to be coated. This means that the minimum film-forming temperature of the coating solution must be lower than the expected drying temperature to be used during device fabrication.

(2) The formed polymer film must be flexible and adherent enough to conform without rupture to the bending and twisting of the coated device under the expected conditions of use.

(3) When the coated device is immersed for long periods in aqueous media such as human blood, the film must not weaken or lose its integrity.

(4) The coating must present a non-cytotoxic and blood compatible surface. When contacted with human blood the coating must not initiate blood coagulation and the complement pathways.

(5) The coating must present a hydrophilic surface and be firmly and securely bound to itself and to the substrate so that no particles or fragments or leachable components can contaminate an aqueous medium such as human blood.

(6) The coating must withstand some acceptable form of sterilization without loss of integrity, durability, biocompatibility, or lubricity.

A coating which satisfies the above requirements is made as described below.

The coatings of the present invention have three chemical characteristics, namely 1) the composition of the top-coat, which generates a lubricious and biocompatible external surface on the composite coating, and 2) the chemical composition of the acrylic copolymer or polyurethane (the “base-coat”) to be used in coating the substrate, and 3) the top-coat is covalently attached to the base-coat, which provides durable and abrasion-resistant coating.

These characteristics are discussed in order, below.

The top-coat includes a hydrophilic polymer that is synthesized by polymerization of hydroxyl group-containing ethylenic monomers, acidic group-containing ethylenic monomers and other monomers containing hydrophilic functional groups, and a portion of the acid groups may be neutralized.

The copolymer of the present invention is prepared by copolymerizing 100 to 5% by weight of non-ionizable hydrophilic ethylenic monomers, 0 to 95% by weight of acidic group-containing ethylenic monomers, and the balance of ethylenic monomers other than acidic group-containing ethylenic monomers and hydroxy group-containing ethylenic monomers. The non-ionizable, hydrophilic monomers may be hydroxyl-group containing ethylenic monomers or aprotic, hydrophilic ethylenic monomers.

The hydroxylic group-containing ethylenic monomers are selected from the group consisting of N-(2-hydroxyethyl)acrylamide, 2-hydroxyethyl methacrylate, 2-hydroxypropyl acrylate, 2-hydroxypropyl methacrylate, 2,4-dihydroxy-4′-vinyl benzophenone, and N-(2-hydroxyethyl)methacrylamide, N-acryloylamido-ethoxyethanol, N-(hydroxymethyl)acrylamide, N-[tris(hydroxymethyl)methyl]acrylamide, 4-hydroxybutyl acrylate, hydroxypropyl acrylate, methyl 3-hydroxy-2-methylenebutyrate, hydroxypropyl methacrylate, 2-allyloxyethanol, 3-allyloxy-1,2-propanediol, 1,4-butanediol vinyl ether, di(ethylene glycol)vinyl ether, ethylene glycol vinyl ether, N,N-1,2-dihydroxyethylene-bis-acrylamide, N,N-1,2-dihydroxyethylene-bis-methacrylamide, N-hydroxymethyl methacrylamide, N-tri(hydroxymethyl)-methyl-methacrylamide, or a mixture thereof.

The aprotic, hydrophilic monomers may be N-vinyl pyrrolidone, acrylamide and its N-alkyl derivatives, Poly(ethylene glycol) (n) monomethyl ether monomethacrylate, 2-methacryroyloxyethylphosphorylcholine, [3-(Methacryloylamino)propyl]dimethyl(3-sulfopropyl)ammonium hydroxide inner salt, [2-(Methacryloyloxy)ethyl]dimethyl-(3-sulfopropyl)ammonium hydroxide.

The acid group-containing ethylenic monomers are selected from the group consisting of acrylic acid, methacrylic acid, 2-Ethylacrylic acid, 2-Propylacrylic acid, acryloxypropionic acid, isocrotonic acid, maleic anhydride, maleic acid and half esters, half amides and half thioesters of maleic acid, fumaric acid and itaconic acid, or a mixture thereof.

The weight average molecular weight of the invented copolymer may be 50,000-10,000,000 Daltons. Preferably, the weight average molecular weight is 100,000 to 1,000,000 Daltons. Most preferably, the weight average molecular weight of the carboxylic acid containing, hydrophilic polymer is 200,000 to 800,000 Daltons.

The copolymer of the present invention may be prepared by free radical polymerization, atom transfer radical polymerization, anionic polymerization, and other suitable polymerization methods. The free radical polymerization can be initiated by redox, thermal and photo initiators.

The invented copolymer, which comprise hydroxyl, hydrophilic aprotic, and acid groups, is highly hydrophilic and thus provides great wet lubricity when used as coating for a device or substrate. When contacted with human blood, carboxylic acid containing polymers may show material mediated coagulation abnormalities in the intrinsic pathway. The introduction of hydroxylic or hydrophilic aprotic groups here disclosed by this invention is proven to greatly improve blood-compatibility of the polymer using as a coating or hydrogel.

The copolymer of the present invention may be formulated into a coating. The pH of the coating solution can be from 2.3 to 10.0. The preferred pH of the top-coat solution is from 3.5 to 5.0.

The coating solution is applied after a base-coat has dried and formed a water insoluble coating layer. After the coating has been applied, the base-plus-top coated materials are baked in an oven at 50-120° C. to fully cure the base-coat and top-coat.

The coating prepared from the copolymer of the present invention demonstrates a combination of good lubricity, durability to wear and is non-thrombogenic. Those are critical properties for successful friction-reducing medical coatings.

The utilization of polymers containing hydroxyl, hydrophilic aprotic, and carboxylic acid groups in this application is a novel improvement over the prior art. In particular, the unique chemistry of the coating ensures the combination of excellent non-thrombogenicity, lubricity and durability. The synthetic polymer coating also possesses exceptional resistance to gamma and e-beam radiation sterilization procedures. After gamma or e-beam sterilization, the hydrophilic coating retains its lubricity and durability. The coating system is also proven to be non-cytotoxic by the MEM Elution test.

In the coating of the present invention, a hydrophilic coating provides lubricity for the coated medical devices when contacted with aqueous media. The base-coat used is an intermediate layer between the functional hydrophilic top-coat and the medical device surface. The base-coat possesses good adhesion to the medical device substrate. Suitable base-coats can be acrylic polymers, polyurethanes, or acrylate-urethane copolymers. The useful base-coats are acrylic polymers, polyurethane dispersions and acrylate-urethane copolymers, which are reactive with polyfunctional isocyanate and/or polyfunctional aziridines. The isocyanate and aziridine compound in the base-coat formulation is utilized to graft the top-coat polymers to the base-coat polymer. Therefore the hydrophilic polymer in the top-coat is chemically attached to the base-coat layer. The cured base-coat absorbs a very small amount of water, so it can maintain its adhesion when the coated medical devices are used in aqueous media. At the same time the top-coat, which is fixed on the base-coat, is ready to be hydrated and provides lubricity.

In the case of an acrylic base-coat, the coating can be either a solvent-based acrylate polymer solution or an aqueous acrylate polymer colloidal dispersion. The base-coat will normally be formulated with a polyfunctional crosslinking agent, such as a polyfunctional aziridine or a polyfunctional isocyanate or both. The polyfunctional compound in the base-coat is not only used to cross-link the base-coat polymer, but also to react with top-coat polymer at the interface and tie the two coats together by chemical bonding.

The acrylic polymer base-coat can be solvent-based and will include one or more functional groups selected from hydroxylic monomers, such as hydroxyethyl methacrylate, and acidic monomers, such as acrylic acid. The cross-linking and grafting agents can be polyfunctional isocyanates or polyfunctional aziridines or both.

Suitable acrylic polymer base-coats described above include those supplied under the HYDAK trademark, specifically HYDAK B-23K, HYDAK B-500, HYDAK S-103, and HYDAK DC-8. HYDAK is a trademark of Biocoat, Inc. HYDAK B-23K is a base-coat that contains hydroxyl functionality and is suitably cross-linked with a polyisocyanate. HYDAK B-500 and HYDAK S-103 are base-coats that contain both hydroxyl and carboxylic acid functionality and are suitably cross-linked by a mixture of a polyfunctional aziridine and a polyfunctional isocyanate. HYDAK DC-8 is a base-coat that contains carboxylic acid functionality and is suitably cross-linked with a polyfunctional aziridine. Suitable base-coats may also be acrylic coating solutions or water dispersions provided by other suppliers that are capable of reacting with polyfunctional aziridines. Mixtures of acrylic base-coats also may be suitable.

Suitable base-coats can also be polyurethane dispersions in water. Such polyurethanes comprise those with built-in organic acid groups that are reactive to polyfunctional aziridines. Preferred organic acid groups are carboxylic acids or their partially neutralized salts. The polyfunctional aziridine in the base-coat is not only used to cross-link the base-coat polymer, but also to react with the top-coat polymer at the interface and tie the two coats together by chemical bonding. By this means, a top-coat containing carboxylic acid groups can be grafted on the base-coating layer. Examples of suitable polyurethane base-coats include those supplied: under the trademarks NeoRez R1010, NeoRez R551, NeoRez R563, NeoRez R600, NeoRez R940, NeoRez R960, NeoRez R9621, NeoRez R9637, NeoRez R967, NeoRez R9679, and NeoRez R974, NeoRez being a trademark of DSM, and under the trademarks Sancure 20040, Sancure 20037F, Sancure PC-52, Sancure 1049C, Sancure 11525, Sancure 12929, Sancure 12954, Sancure 13094HS, Sancure 20025, Sancure 777F, Sancure 815, Sancure 815D, Sancure 777, Sancure 825, Sancure 898, and Sancure 20041, Sancure being a trademark of Lubrizol.

Another specific class of polymers which may be used as the base-coat for the hydrophilic coating described in this disclosure are acrylate-urethane hybrids, for example those supplied under the trademarks NeoPac 9699 by DSM and Sancure AU 4010 by Lubrizol.

The base-coats described above are used with polyfunctional aziridines. Examples of such polyfunctional aziridines are supplied under the trademarks Neocryl CX-100 by DSM and XAMA-7 by Bayer A G. The base-coat formulation may also include suitable polyfunctional isocyanates as cross-linking agents in addition to polyfunctional aziridines. One example of such a polyfunctional isocyanate is Desmodur N75.

The description above introduces an improved polymer coating or hydrogel system in which a new hydrophilic polymer is invented. When used, the polymer coating or hydrogel imparts non-thrombogenic surface and greatly reduces the friction of medical devices when they are inserted and passed over tissues and, therefore, prevents mechanical abrasion and discomfort.

The invention will be further illustrated in the following non-limiting examples representing presently preferred embodiments of the invention.

### EXAMPLE 1

This Example describes the preparation of poly(N-hydroxyethyl acrylamide-co-acrylic acid) 75/25 by mol.

Reagents used in the polymerization process are N-(2-hydroxyethyl)acrylamide (HEAA), acrylic acid (AA), ammonium persulfate (APS), sodium hydroxymethanesulfinate hydrate (SHMS) and ferrous sulfate heptahydrate (FeSO₄ 7H₂O) and high purity water. All the reagents except water are purchased from Sigma-Aldrich. 33.10 g of HEAA and 6.90 g of AA are added into 360 g of high purity water. 0.10 g of APS and 0.065 g of SHMS are used to initiate the polymerization. 0.05 mL of 1% FeSO₄ is used as a catalyst. The polymerization is conducted at 40° C. under nitrogen with stirring. The monomer conversion and GPC analysis are conducted for the polymerization products. The results are shown in Table 1. The conversion is measured by drying polymer product solution at 60° C. for 2 hours and then calculated by the equation as follows:

$$conversion ( \% ) = \frac{( weight of solids after drying )}{( polymer weight at 100 \% conversion )} \times 100$$

The greater than 100× conversion is probably the result of incomplete drying of the polymer product.

TABLE 1
Results of GPC analysis of polymerization Products

| | |
|---|---|
| Measured Monomer | GPC analysis |

| Lot No. | Conversion (%) | Mₙ | M_{w} | M_{w}/Mₙ |
|---|---|---|---|---|
| A | 105.0 | 87,300 | 381,000 | 4.36 |
| B | 104.3 | 76,300 | 391,000 | 5.13 |
| C | 105.3 | 90,200 | 436,000 | 4.83 |

Mₙ is the number-average molecular weight, and M_{w} is the weight-average molecular weight.

### EXAMPLE 2

This Example relates to the preparation of poly(N-hydroxyethyl acrylamide-co-acrylic acid) 50/50 by mol.

24.00 g of HEAA and 15.01 g of AA are added into 261 g of high purity water. 0.0672 g of APS and 0.0430 g of SHMS are used to initiate the polymerization. The polymerization was conducted at 60° C. under nitrogen with sufficient stirring. The monomer conversion of the polymerization was 104% and the polymer product of 7.80% solids had a viscosity of ‘U’ measured by Gardner bubble viscometer.

### EXAMPLE 3

This Example concerns the preparation of poly(N-hydroxyethyl acrylamide-co-acrylic acid) 95/5 by mol.

37.76 g of HEAA and 1.24 g of AA are added to 261 g of high purity water. 0.0799 g of APS and 0.0520 g of SHMS are used to initiate the polymerization. The polymerization was conducted at 60° C. under nitrogen with sufficient stirring. The monomer conversion of the polymerization was 99.4% and the polymer product of 7.46% solids had a viscosity of ‘G’ measured by Gardner bubble viscometer.

### EXAMPLE 4

This Example concerns the preparation of poly(N-vinyl pyrrolidone-co-acrylic acid) 50/50 by mol.

N-vinyl pyrrolidone (NVP) was purchased from Aldrich and distilled before use. 17.70 g of AA was used and 20% NH₄OH was added to adjust the pH to 5.0. The pH adjusted AA and 27.30 g of NVP were added to 255 g of high purity water. 0.48 g of 1% FeSO₄ solution and 0.24 g of 70% t-butyl hydroperoxide solution (Aldrich) are used to initiate the polymerization. The polymerization was conducted at 40° C. under nitrogen with sufficient stirring. The monomer conversion of the polymerization was 91.3% and the polymer product of 6.85% solids had a viscosity of ‘H’ measured by Gardner bubble viscometer.

### EXAMPLE 5

In this Example, several different bilaminar coatings were applied to copolyester (PETG) rods. The PETG rods of ⅛ inch diameter were purchased from McMaster Carr. The rods were wiped with isopropyl alcohol to clean the surface and allowed to dry before applying the coating.

A base-coat was prepared by adding the ingredients successively to a beaker under proper agitation until thoroughly mixed. HYDAK B-500 is an acrylic polymer solution in PM acetate, manufactured by Biocoat, Inc. Polyisocyanate and polyaziridine were used as curing agents.

Top-coat formulations were prepared by mixing the hydrophilic polymers prepared in Examples 1-4 according to the formulations recorded in Tables 2. A poly(acrylic acid) (PAA) formulation was included as a control. Each formulation included suitable additives and cross-linkers. The PAA has a molecular weight of 450,000 Daltons and was purchased from Polysciences, Inc. Chromium (III) sulfate and CX-100 were used as cross-linkers and purchased from Aldrich. All the solutions used for top-coat were prepared by using water as the solvent.

Both base-coat and top-coat were applied to PETG rods by a dip-coating method at a withdrawal speed of 0.2 inch per second. A thin layer of coating solution remains on the substrate surface. The samples were dried in an oven at 60° C. for 20 minutes after base-coat was applied. The top-coat was then applied and the bilaminar coating cured for 2 hours at 60° C. The cured samples were washed with 0.5% (wt/wt) NaHCO₃ and followed by washing with water and drying at 60° C. for 20 minutes. The dried samples were then used for performance testing.

TABLE 2
Coating composition and application on PETG rods

| Sample ID | Top-coat | Base-coat |
|---|---|---|
| PAA | 2.5% wt PAA (MW 450,000) | HYDAK B-500 with |
| | with additives | cross-linkers |
| P(HEAA-AA) | 2.5% wt P(HEM-M) 50/50 | HYDAK B-500 with |
| 50/50 | by mol (prepared in | cross-linkers |
| | Example 2) with additives | |
| | and cross-linkers | |
| P(HEAA-AA) | 2.5% wt P(HEAA-AA) 75/25 | HYDAK B-500 with |
| 75/25 | by mol (prepared in | cross-linkers |
| | Example 1) with additives | |
| | and cross-linkers | |
| P(HEAA-AA) | 2.5% wt P(HEM-AA) 95/5 | HYDAK B-500 with |
| 95/5 | by mol (prepared in | cross-linkers |
| | Example 3) with additives | |
| | and cross-linkers | |
| P(NVP-AA) | 2.5% wt P(NVP-AA) 50/50 | HYDAK B-500 with |
| 50/50 | by mol (prepared in | cross-linkers |
| | Example 3) with additives | |
| | and cross-linkers | |

### EXAMPLE 6

In this example the samples with different coatings from Example 5 were tested for their thrombogenic performance. The partial thromboplastin time (PTT) test was conducted by Nelson Labs. The PTT test was used as a general screening test for the detection of material mediated coagulation abnormalities in the intrinsic pathway. The assay was developed as a modification to the plasma re-calcification time test with the variable of platelet concentration being controlled by the addition of a phospholipid platelet substitute (PTT Reagent) to platelet poor plasma.

Human blood was drawn using vacutainers containing 0.1 M sodium citrate at a ratio of 9:1 (blood to anticoagulant). The blood was maintained refrigerated and used within 4 hours of blood draw. The test articles were prepared by exposing 6 cm² of the test article in 2.0 mL of plasma. Polypropylene pellets (0.4 grams/2.0 mL) were tested as the negative control. Plasma alone was also tested. Approximately 6.0 cm² glass beads were exposed in 2.0 mL of plasma and included as a positive control. The test articles and controls were exposed to the plasma at room temperature for 60 minutes.

Following exposure, 0.2 mL aliquots of the plasma were transferred to individual test tubes. Six replicates of the test article and controls were prepared. The test tubes were placed into a water bath and incubated for 60 seconds at 37+/−1° C. A 0.2 mL aliquot of the PTT reagent was added to each test tube and incubated for 3 minutes. Calcium chloride (0.2 mL) was added and the time required for the plasma to clot determined. The procedure was repeated for a total of six replicates. Clotting time was recorded in seconds. Averages and standard deviations were calculated for the test article and controls.

Because of its proven non-thrombogenicity, HYDAK L110 (Biocoat, Inc.), a sodium hyaluronan coating, was used as the predicate for the PTT test.

The PTT test results are shown in Table 3. Statistical analysis of the test results was conducted by Nelson Labs based on the MINITAB program. Statistical analysis of the data indicated that there was no statistically significant difference between the clot time of the predicate (HYDAK L110) and the negative control. There was a statistically significant difference between the article coated with PAA coating and the negative control, and also between the negative control and the articles coated with P(HEAA-AA) 50/50, P(HEAA-AA) 95/5 and P(AA-NVP) 50/50 coating. Those articles coated with PAA, P(HEAA-AA) 50/50, P(HEAA-AA) 95/5 and P(AA-NVP) 50/50 demonstrated a shortened clot time when compared to the negative control. The magnitude of the difference between the coatings of copolymer and the negative control was much smaller than that of PAA coating and the negative control. It should be noted that there was no statistically significant difference between the clot time of the article coated with P(HEAA-AA) 75/25 coating and the negative control. The results indicate that P(HEAA-AA) 75/25 coating has the lowest possibility to cause material mediated coagulation when it contacts with human blood.

Based on the PTT test, incorporation of either HEAA or NVP into the AA polymer has greatly improved its anti-thrombogenic performance. The P(HEAA-AA) copolymer with a HEAA/AA molar ratio of 75/25 had the best performance in the PTT test.

TABLE 3
Performance of different coatings in the
partial thromboplastin time (PTT) test

| Coating | PTT (positive, negative control), seconds |
|---|---|
| HYDAK L110 | 159 (67, 159) |
| PAA | 84 (67, 159) |
| P(HEM-AA), 50/50 | 204 (109,224) |
| P(HEAA-AA), 75/25 | 224 (109,224) |
| P(HEAA-AA), 95/5 | 211 (109,224) |
| P(AA-NVP), 50/50 | 202 (109,224) |

### EXAMPLE 7

In this example the friction properties and coating durability of the samples prepared in Example 5 were tested in a pinch tester.

The pinch tester is a device designed for measuring the performance of lubricious coatings on medical devices such as catheters, guide wires, and similar products. The tester has two pads and a mechanism that can apply an adjustable pinch force that clamps these two pads together. A sample, such as a piece of catheter, is pulled through two pads. The pulling rate is controlled by a digital force tester (Chatillon TCD225). The digital force tester is also used to measure and record the pulling force, which is essentially the friction between the sample surface and the pads.

The test was conducted while the pads and the tested segment of the sample were all submerged in 37° C. phosphate-buffered saline. An appropriate length of sample was pulled through the clamped pads. Then the sample was pushed back to the starting position through the clamped pads so that the test could be repeated. The frictional force measured when the sample was pushed through the clamped pads was usually similar to that measured when the sample was pulled through the pads. The digital force tester records both static friction (the initial value when the test was started) and dynamic friction (the amount of friction as the test sample was in motion). When repeated cycles of testing were conducted, the growth of the dynamic friction during the test was used as an indicator of the durability of the coating. A smaller rate of growth of friction indicates a more durable coating.

The pinch test was conducted under pinch forces of 770, 1070 or 1370 grams and the length of sample tested was 3 inches. For each test cycle the dynamic friction was the average value of the frictional force of the entire tested length. Each sample was tested for 100 cycles through the pinch tester. Six samples were tested for each formulation.

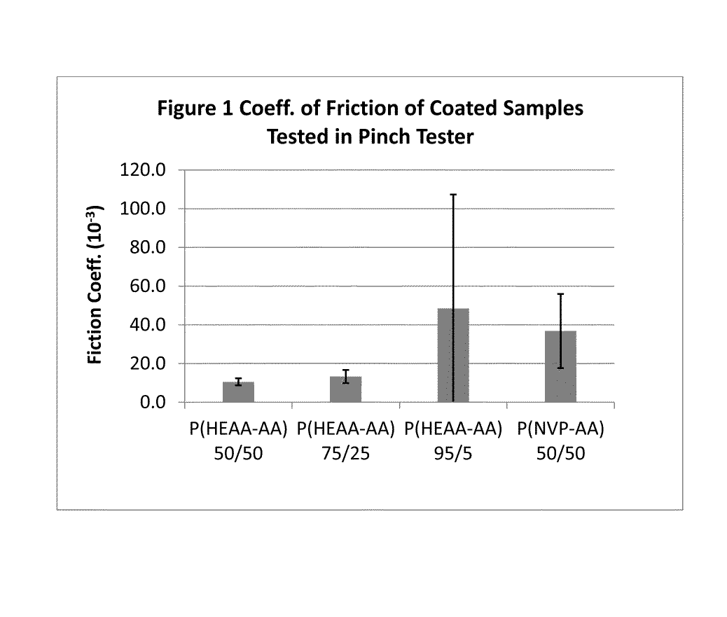

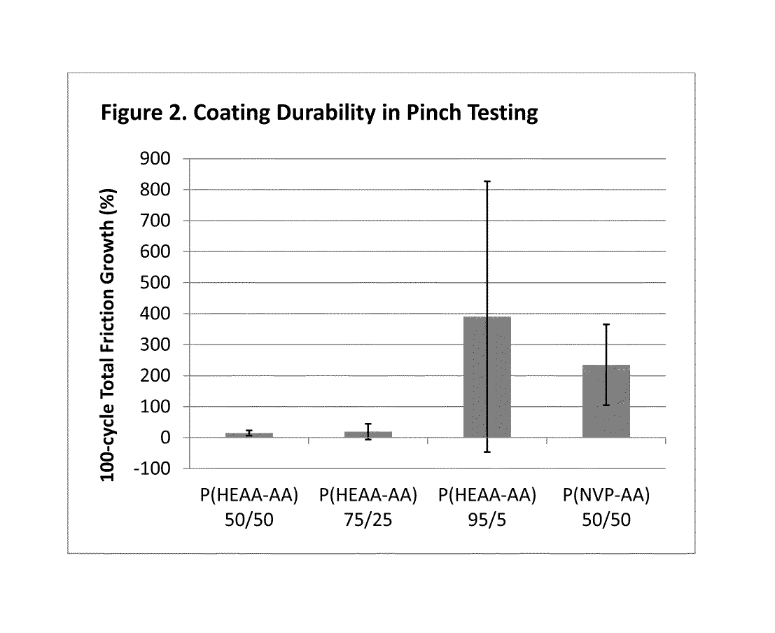

The pinch test results of samples prepared in Example 5 other than PAA coating are shown in FIGS. 1 and 2. The dynamic friction coefficient and its total growth after 100 cycles in FIG. 1 were the average of six samples (n=6). The coating of P(HEAA-AA) 50/50 and P(HEAA-AA) 75/25 had coefficient of friction of 0.010 and 0.013, respectively, compared with a value of 0.30 for uncoated rods. These two top coats also showed good durability with total friction increase of 15% and 19% after 100 consecutive runs in pinch testing. P(HEAA-AA) 95/5 and P(NVP-AA) 50/50 coatings have a relatively poorer durability and higher average friction coefficient.

### EXAMPLE 8

In this Example, samples were prepared by applying P(HEAA-AA) 75/25 top-coat solution and HYDAK B-500 base-coat solution to PETG rods. Three batches of coated samples were prepared, as shown in Table 4.

Both base-coat and top-coat were applied to PETG rods by a dip-coating method at a withdrawal speed of 0.2 inch per second. A thin layer of coating solution remained on the substrate surface. The samples are dried in an oven at 60° C. for 20 minutes after the base-coat is applied. The top-coat is then applied and the bilaminar coating is cured for 1 hours at 60° C.

Fifty-five 18-inch-long rods were coated and cured. Twenty-seven of the cured samples were washed with 0.5% (wt/wt) NaHCO₃ and water, and dried at 60° C. for 20 min. The remaining twenty-eight samples were not washed.

TABLE 4
Preparation of P(HEAA-AA) 75/25 coating on PETG rods

| Lot No. | Top-coat | Base-coat |
|---|---|---|
| JL100511 | 2.5% wt P(HEAA-AA) 75/25 | HYDAK B-500 with |
| | by mol with additives | cross-linkers |
| JL100611 | 2.5% wt P(HEAA-AA) 75/25 | HYDAK B-500 with |
| | by mol with additives | cross-linkers |
| JL101211 | 2.5% wt P(HEAA-AA) 75/25 | HYDAK B-500 with |
| | by mol with additives | cross-linkers |

### EXAMPLE 9

In this Example, the cytotoxicity of a bilaminar coating prepared in Example 8 was tested, in which P(HEAA-AA) 75/25 was used as the top-coat.

The coated samples were evaluated by Nelson Laboratories for cytotoxicity by the MEM Elution Test. The results are shown in Table 5. Both the washed and unwashed samples made according to Example 8 were reported to be non-cytotoxic.

TABLE 5
Cytotoxicity test of P(HEAA-AA) 75/25 Coating

| | | MEM | | | | | MEM |
|---|---|---|---|---|---|---|---|
| | Treatment | extraction | | MEM test Scores | | | Results |
| Sample | after cure | ratio | | #1 | #2 | #3 | (Pass/Fail) |
| JL100611-2 | Washed | 3 | cm²/mL | 0 | 0 | 0 | Pass |
| JL100611-54 | Unwashed | 3 | cm²/mL | 0 | 0 | 0 | Pass |
| Negative | N/A | 0.2 | g/mL | 0 | 0 | 0 | N/A |
| control | | | | | | | |
| (PP pellets) | | | | | | | |
| Positive | N/A | 0.2 | g/mL | 4 | 4 | 4 | N/A |
| control | | | | | | | |
| (Latex | | | | | | | |
| Natural | | | | | | | |
| Rubber) | | | | | | | |

### EXAMPLE 10

In this Example, the performance of the bilaminar coating according to Example 8 was tested after gamma-ray sterilization, in which P(HEAA-AA) 75/25 was used as the top-coat. The performance the sterilized samples in accelerated aging was also tested. The accelerated aging was conducted in a 52° C. oven with ventilation.

Unwashed samples from Example 8 were gamma-ray sterilized and subjected to accelerated aging. The sterilization was conducted as an engineering run by Steris Isomedix using a regular dose for medical devices (approximately 25 kGy). The properties of the coating were evaluated after gamma-ray sterilization.

The sterilized and aged samples were tested in the pinch tester as described in Example 7. Three batches of top coat were tested and four samples from each batch. Pinch forces used for the test were 770 gram and 1070 gram, respectively. Fifty test cycles were conducted for each sample. The dynamic friction of each sample was the average of those of 50 cycles. The growth of friction coefficient after 50 testing cycles was also calculated as the indicator of coating durability.

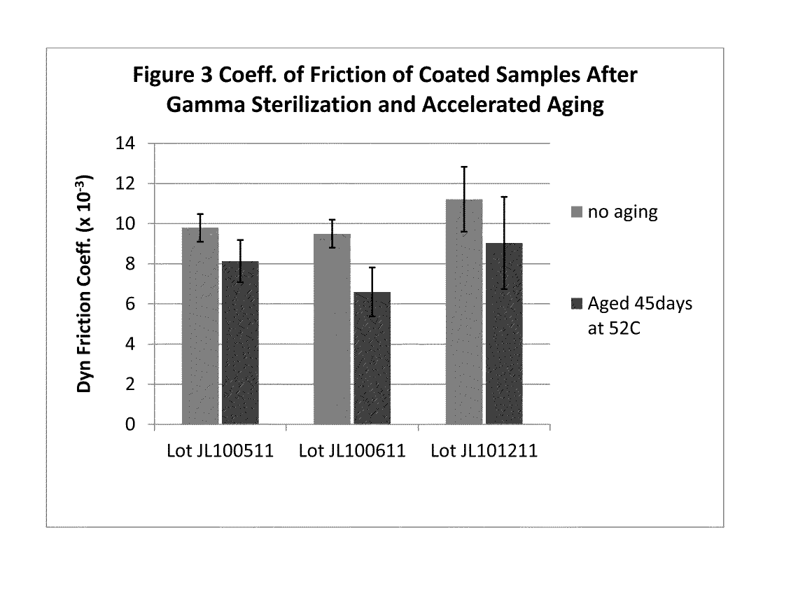

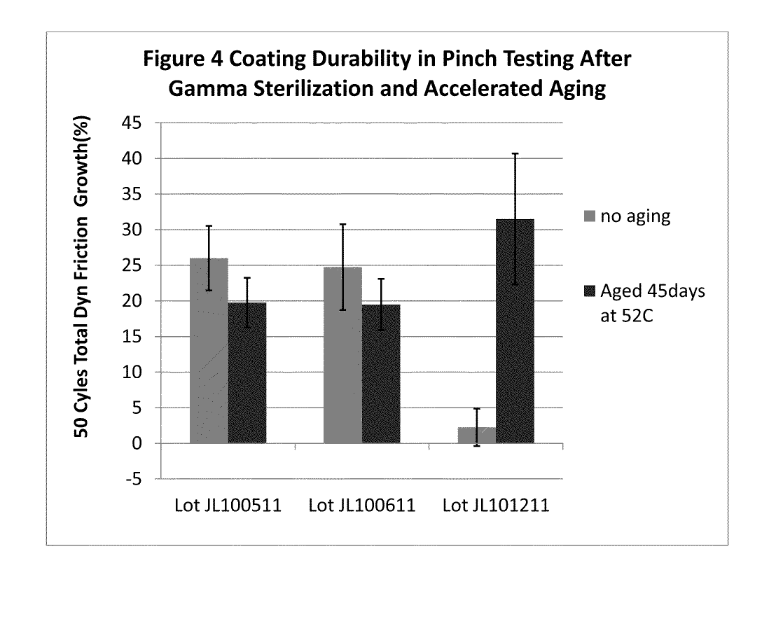

The pinch test results are shown in FIGS. 3 and 4. The friction coefficient and its growth shown on the graphs for each batch were the average of the four samples tested. As shown in FIG. 3, the coefficient of friction of P(HEAA-AA) coating after gamma ray sterilization was in the range of 0.008 to 0.013. A dramatic reduction of friction coefficient was achieved compared with uncoated samples which had coefficient of friction of 0.3.

The aging performance of the gamma-ray sterilized coating was also tested. As shown in FIG. 3, the coating friction coefficient after 45 days at 52° C. was in the range of 0.006 to 0.011. Combining all data of the three batches, a linear regression of friction coefficient was conducted on its dependence on aging time. The results show that the friction coefficient decreases by 5.0×10⁻⁵ (or about 0.5%) per day when aged at 52° C. A statistical T-test shows the decrease of friction with aging time is statistically significant at the 5% confidence level (t value of 3.70 and degree of freedom of 22), although the rate of change with aging is small.

The coating durability is shown in FIG. 4. The gamma-sterilized samples had a total friction growth of less than 30% after 50 cycles in pinch testing. The pinch testing results indicated that the gamma sterilized coating has good durability under high load abrasion. After accelerated aging at 52° C. for 45 days, the total friction growth after 50 testing cycles was in the range of 10 to 40%. Combining all data of the three batches, a linear regression and statistical test shows that there was no statistically significant correlation between coating durability and accelerated aging at 5% level (t value of 1.43 and 22 degrees of freedom). In other words, there is no statistical difference of coating durability between aged and un-aged samples.

Pinch testing results of three different batches demonstrated that the P(HEAA-AA)/HYDAK B-500 coating can withstand gamma irradiation sterilization. The coating retained its lubricity and durability after gamma irradiation sterilization. The sterilized coating also showed its stability in accelerated aging for at least 45 days at 52° C., an equivalent of one year at 22° C.

### EXAMPLE 11

In this Example, the performance of the bilaminar coating according to Example 8 was tested after E-Beam sterilization, in which P(HEAA-AA) 75/25 was used as the top-coat. The performance of the sterilized samples in accelerated aging was also tested. The accelerated aging was conducted in a 52° C. oven with ventilation.

Unwashed samples of Example 8 were used for E-Beam sterilization and accelerated aging. The sterilization was conducted by BeamOne, LLC using a dose of 25 kGy+/−10%. The properties of the coating were evaluated after E-Beam sterilization.

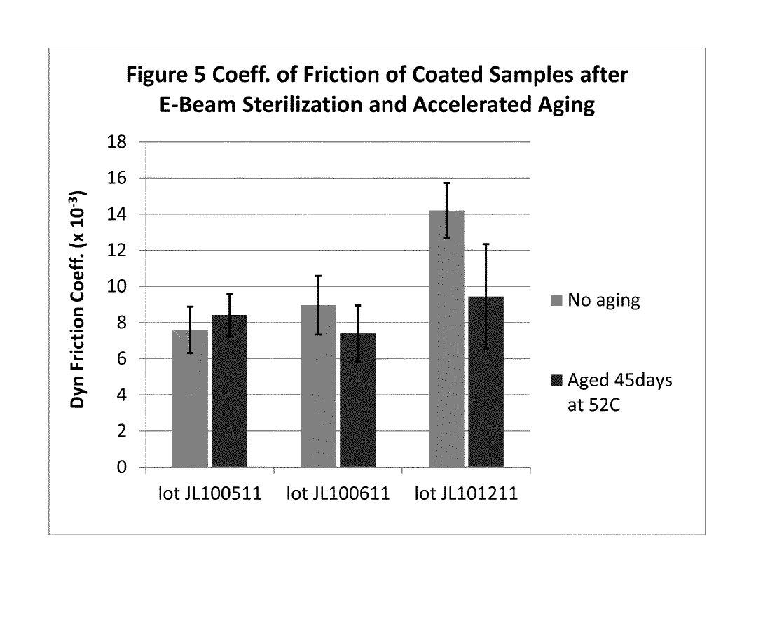

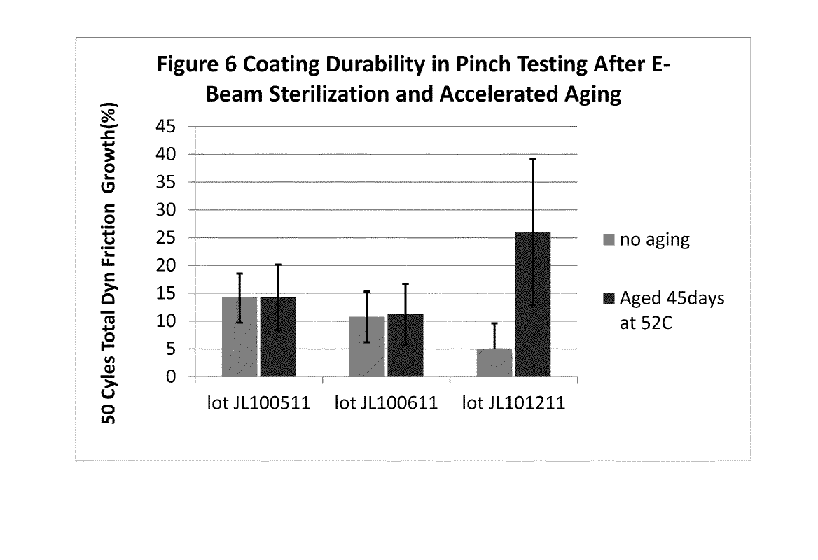

The sterilized and aged samples were tested in the pinch tester as described in Example 8. Three batches of top coat were tested. Four samples of coated rods were taken from each batch of top coat. The pinch forces used for the tests were 770 gram and 1070 gram, respectively. Fifty cycles were conducted for each sample. The dynamic friction of each sample was the average of those 50 cycles. The growth of the friction coefficient after 50 testing cycles was also calculated as the indicator of coating durability. The pinch test results are shown in FIGS. 5 and 6.

The coefficient of friction and its growth were averaged for the four samples from each batch tested. As summarized in FIG. 5, the friction coefficient of P(HEAA-AA) coating after E-beam sterilization was in the range of 0.006 to 0.016. A dramatic reduction of friction coefficient was achieved compared with uncoated samples which had a coefficient of friction of 0.3.

The aging performance of the E-Beam- sterilized coating was also tested. As shown in FIG. 5, the coating friction coefficient after 45 days at 52° C. was in the range of 0.006 to 0.012. Combining all data of the three batches, a linear regression of friction coefficient was conducted on its dependence on aging time. A statistical T-test shows that there was no statistically significant correlation between friction coefficient and accelerated aging at the 5% confidence level (t value of 1.66 and 22 degrees of freedom). In other words, there is no statistical difference of friction coefficient between aged and un-aged samples.

The coating durability is summarized in FIG. 6. The E-Beam sterilized samples have a total friction growth of less than 20% after 50 cycles in pinch testing, comparable to 19+/−20% total growth before sterilization. After accelerated aging at 52° C. for 45 days, the total friction growth after 50 testing cycles was in the range of 5 to 40%. Combining all data of the three batches, a linear regression of friction coefficient was conducted on its dependence on aging time. The percentage of total 50-cycle frictional growth increased by 0.16 per day when aged at 52° C. A statistical T-test shows that the correlation between durability and aging time is statistically significant at the 5% confidence level (t value of 2.09 and degree of freedom of 22), although the value of the durability change with aging is very small.

Pinch testing results of three different batches demonstrates that the P(HEAA-AA)/HYDAK B-500 coating can withstand E-Beam irradiation sterilization. The coating retains its lubricity and durability after E-Beam sterilization. The sterilized coating also shows its stability in accelerated aging for at least 45 days at 52° C., an equivalent of one year at 22° C.

### EXAMPLE 12

In this example, the performance of the bilaminar coating according to Example 8 was tested after ethylene oxide (ETO) sterilization, in which P(HEAA-AA) 75/25 was used as the top-coat. The performance of the sterilized samples in accelerated aging was also tested. The accelerated aging was conducted in an oven at 52° C. with ventilation.

Unwashed samples from Example 8 were used for ETO sterilization and accelerated aging. The sterilization was conducted by Anderson Scientific exposing the samples to ETO for greater than 16 hours of sterilization at less than 33° C. The properties of the coating were evaluated after ETO sterilization.

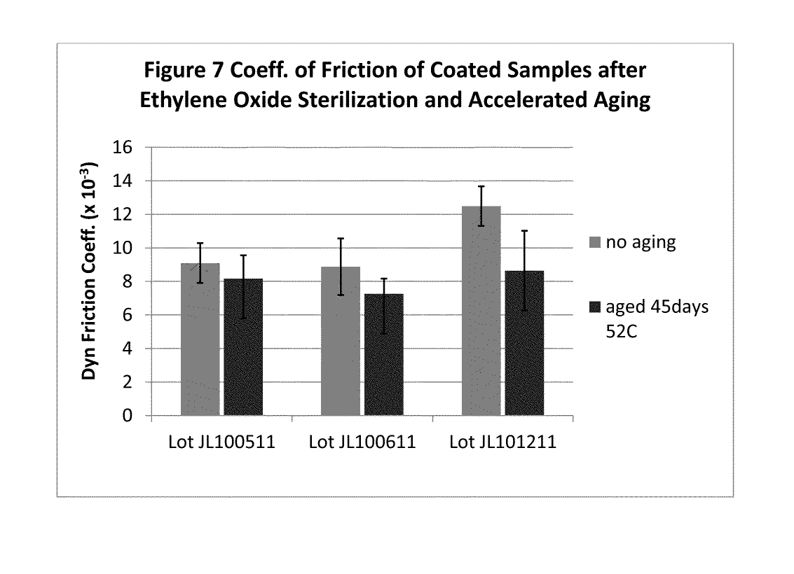

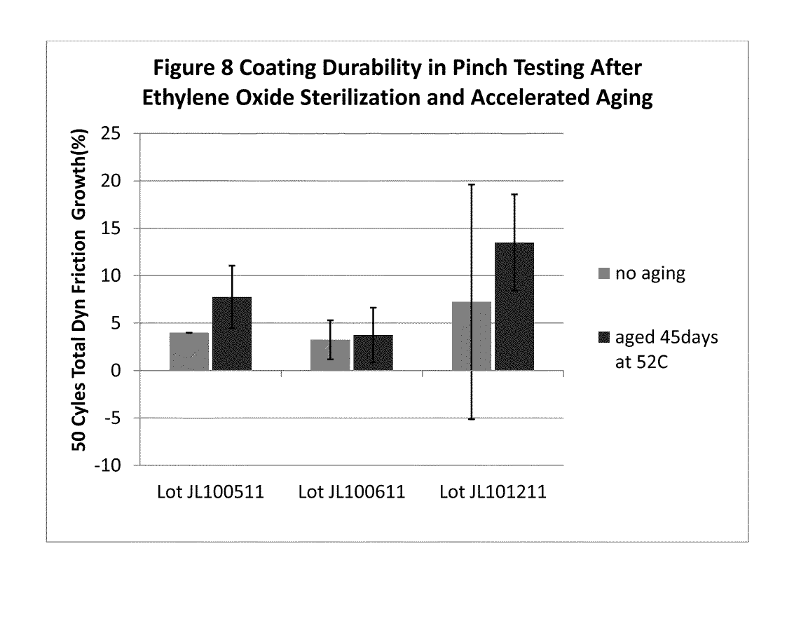

The sterilized and aged samples were tested in the pinch tester as described in Example 7. Three batches were tested with four samples from each batch. The pinch forces used for each batch were 770 grams and 1070 grams, respectively. Fifty test cycles were conducted for each sample. The dynamic friction of each sample is the average of those of 50 cycles. The growth of the friction coefficient after 50 testing cycles was also calculated as an indicator of coating durability. The pinch test results are shown in FIGS. 7 and 8.

The friction coefficient and its growth shown were averaged for the four samples from each batch tested. As summarized in FIG. 7, the friction coefficient of P(HEAA-AA) coating after ETO sterilization was in the range of 0.007 to 0.014. A dramatic reduction of friction coefficient is achieved compared to uncoated samples which had a friction coefficient of 0.3. The aging performance of the ETO-sterilized coating was also tested.

As shown in FIG. 7, the coefficient of friction for the coating after 45 days at 52° C. is in the range of 0.005 to 0.011. Combining all data of the three batches, a linear regression of friction coefficient was conducted on its dependence on aging time. The results showed that the friction coefficient decreased by 4.7×10⁻⁵ (or about 0.5%) per day when aged at 52° C. A statistical T-test showed that the decrease of friction is statistically significant at 5% confidence level (t value of 2.75 and 22 degrees of freedom), although the change in coefficient of friction with aging is small.

The coating durability is summarized in FIG. 8. The ETO sterilized samples have a total friction growth of less than 20% after 50 cycles in pinch testing comparable to 19+/−20% total growth before sterilization. After accelerated aging at 52° C. for 45 days, the total friction growth after 50 testing cycles was less than 20%. Combining all data of the three batches, a linear regression and statistical test shows that there is no statistically significant correlation between coating durability and accelerated aging at the 5% confidence level (t value of 1.39 and 22 degrees of freedom). In other words, there is no statistical difference of coating durability between aged and un-aged samples.

Pinch testing results of three different batches demonstrates that the P(HEAA-AA)/HYDAK B-500 coating can withstand ETO sterilization. The coating retains its lubricity and durability after ETO sterilization. The sterilized coating also shows its stability in accelerated aging for at least 45 days at 52° C., an equivalent of one year at 22° C.

### EXAMPLE 13

In this Example, the stability of the P(HEAA-AA)/HYDAK B-500 bilaminar coating was tested in a prolonged soaking in 37° C. phosphate buffered saline (PBS) solution.

The coated sample preparation was the same as that in Example 8.

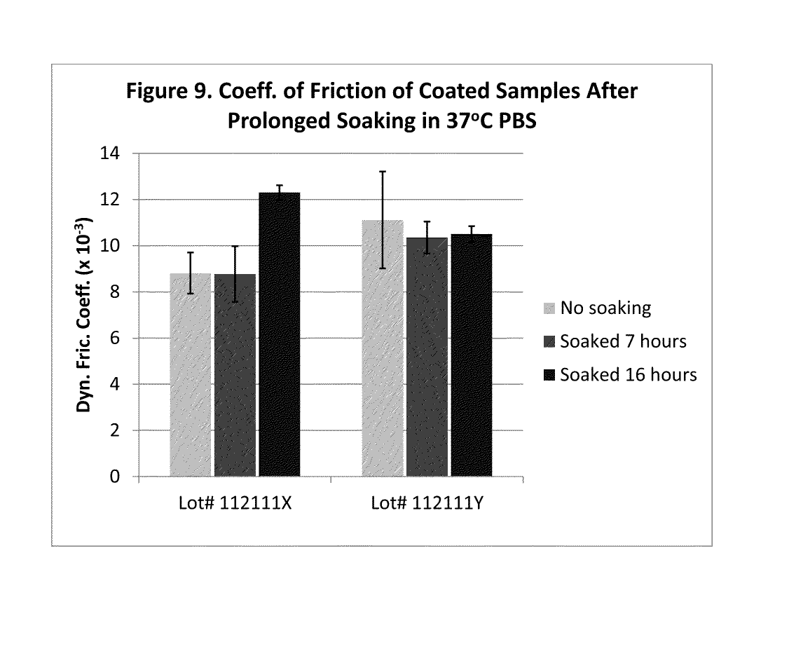

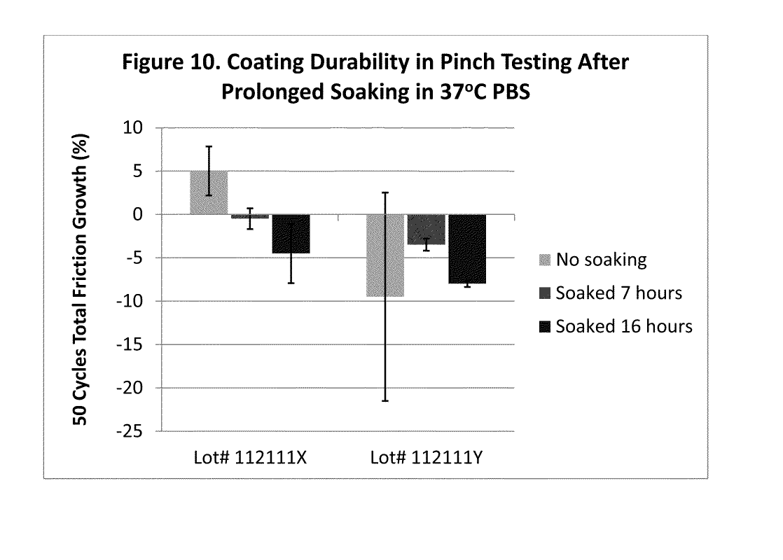

The stability of the coating was tested by soaking the coated PETG rods in 37° C. PBS solution. The soaked samples were then tested immediately by using the pinch tester as described in Example 8. Samples of two batches were tested after soaking for 7 hours and 16 hours in 37° C. PBS solution, respectively. Samples that were not subjected to PBS soaking were also tested as controls. Pinch forces of 770 grams and 1070 grams were used for testing and fifty cycles were conducted for each test. Four samples from each batch were tested. The average friction coefficient was calculated, and so was the total friction growth after 50 cycles. The results are shown in FIGS. 9 and 10.

FIG. 9 showed that the coating friction coefficient after soaking in 37° C. PBS solution for 7 and 16 hours was in the same range as non-soaked samples. Combining the data of two batches, a linear regression and statistical test shows that there was no statistically significant correlation between friction coefficient and time of soaking in PBS at the 5% confidence level (t value of 1.48 and 10 degrees of freedom).

The data in FIG. 10 showed that the total growth of friction after testing for 50 cycles, an indicator of coating durability, is not deteriorated after prolonged soaking in 37° C. PBS solution. Combining the data of two batches, a linear regression and statistical test showed that there is no statistically significant correlation between coating durability and time of soaking in PBS at 5% confidence level (t value of 0.89 and 10 degrees of freedom). The pinch test results demonstrated the coating has good PBS resistance.

The invention can be modified in ways that will be apparent to those skilled in the art. Such modifications should be considered within the spirit and scope of the following claims.

What is claimed is:

1. A coating composition comprising a copolymer formed by copolymerizing 5 to 95% by weight of non-ionizable hydrophilic ethylenic monomers, 5 to 95% by weight of acidic group-containing ethylenic monomers, and a balance comprising other ethylenic monomers.

2. The composition of claim 1, wherein the non-ionizable, hydrophilic monomers are selected from the group consisting of hydroxyl-group containing ethylenic monomers and aprotic, hydrophilic ethylenic monomers.

3. The composition of claim 1, wherein a portion of the acidic group-containing ethylenic monomers is neutralized.

4. The composition of claim 1, wherein said acid group-containing ethylenic monomers are selected from the group consisting of acrylic acid, methacrylic acid, 2-ethylacrylic acid, 2-propylacrylic acid, acryloxypropionic acid, isocrotonic acid, maleic anhydride, maleic acid and half esters, half amides and half thioesters of maleic acid, fumaric acid and itaconic acid, and any mixture of the foregoing.

5. The composition of claim 1, wherein said hydroxylic group-containing ethylenic monomers are selected from the group consisting of N-(2-hydroxyethyl)acrylamide, 2-hydroxyethyl methacrylate, 2-hydroxypropyl acrylate, 2-hydroxypropyl methacrylate, 2,4-dihydroxy-4′-vinyl benzophenone, and N-(2-hydroxyethyl) methacrylamide, N-acryloylamido-ethoxyethanol, N-(hydroxymethyl) acrylamide, N-[tris(hydroxymethyl)methyl]acrylamide, 4-hydroxybutyl acrylate, hydroxypropyl acrylate, methyl 3-hydroxy-2-methylenebutyrate, hydroxypropyl methacrylate, 2-allyloxyethanol, 3-allyloxy-1,2-propanediol, 1,4-butanediol vinyl ether, di(ethylene glycol)vinyl ether, ethylene glycol vinyl ether, N,N-1,2-dihydroxyethylene-bis-acrylamide, N,N-1,2-dihydroxyethylene-bis-methyacrylamide, N-hydroxymethyl methacrylamide, N-tri(hydroxymethyl)-methyl-methacrylamide, and any mixture of the foregoing.

6. The composition of claim 2, wherein the aprotic, hydrophilic monomers are selected from the group consisting of N-vinyl pyrrolidone, acrylamide and its N-alkyl derivatives, poly(ethylene glycol)monomethyl ether monomethacrylate, 2-methacryroyloxyethylphosphorylcholine, [3-(methacryloylamino)propyl]dimethyl(3-sulfopropyl)ammonium hydroxide inner salt, [2-(methacryloyloxy)ethyl]dimethyl-(3-sulfopropyl)ammonium hydroxide.

7. The composition of claim 1, wherein the coating is applied to a device or substrate to provide a hydrophilic and lubricious surface when contacted with aqueous fluid.

8. The composition of claim 1, wherein the coating is applied to a device or substrate to provide non-thrombogenic surface.

9. The composition of claim 1, wherein the coating is applied to a device or substrate to provide a non-fouling surface and resistance to protein and bacteria adsorption.

10. The composition of claim 1, wherein said copolymer is covalently grafted to a primer/base coat, and wherein the primer/base coat is firmly adhered to a substrate.

11. The composition of claim 1, wherein said copolymer is mixed with primer/base coat solutions and dispersions to form a physically entangled structure that is firmly adhered to a substrate.

12. The composition of claim 1, wherein said copolymer is covalently attached to a substrate by photo-radiation.

13. The composition of claim 1, wherein a cross-linking agent is used in the coating formulation.

14. The composition of claim 1, wherein said copolymer is used to form a hydrogel.

15. The composition of claim 7, wherein the coating is sterilized by at least one of gamma-ray, E-beam, and ethylene oxide.

16. The composition of claim 8, wherein the coating is sterilized by at least one of gamma-ray, E-beam, and ethylene oxide.

17. The composition of claim 9, wherein the coating is sterilized by at least one of gamma-ray, E-beam, and ethylene oxide.

18. The composition of claim 10, wherein said primer/base coat is selected from the group consisting of an acrylic polymer solution, an acrylic polymer dispersion, a polyurethane dispersion, an acrylic polyurethane hybrid dispersion, and wherein the primer/base coat comprises at least one of carboxylic acid groups, isocyanate groups, hydroxyl functional groups or their combinations.

19. The composition of claim 11, wherein said primer/base coat is selected from the group consisting of an acrylic polymer solution, an acrylic polymer dispersion, a polyurethane dispersion, an acrylic polyurethane hybrid dispersion, and wherein the primer/base coat comprises at least one of carboxylic acid groups, isocyanate groups, hydroxyl functional groups or their combinations.

20. The composition of claim 10, wherein said primer/base coat contains an active pharmaceutical or antimicrobial agent mixed or blended in with said primer with an intention of releasing said agent from a medical device for therapeutic effect.

21. The composition of claim 11, wherein said primer/base coat contains an active pharmaceutical or antimicrobial agent mixed or blended in with said primer with an intention of releasing said agent from a medical device for therapeutic effect.

22. The composition of claim 20, wherein the pharmaceutical or antimicrobial agent is intended to slowly release out of the hydrogel to provide a therapeutic or antimicrobial effect.

23. The composition of claim 20, wherein the antimicrobial agent is a zeolite containing an antimicrobial ion.

24. The composition of claim 13, wherein the coating is cross-linked by metal ion and ionic polymer.

25. The composition of claim 14, wherein the coating and hydrogel are cross-linked by metal ion and ionic polymer.

26. The composition of claim 13, wherein the coating is cross-linked by photo-radiation.

27. The composition of claim 14, wherein the hydrogel and coating are cross-linked by photo-radiation.

28. The composition of claim 13, wherein the coating is cross-linked by multifunctional aziridine and multifunctional isocyanate compounds.

29. The composition of claim 14, wherein the hydrogel and coating are cross-linked by multifunctional aziridine and multifunctional isocyanate compounds.

30. The composition of claim 14, wherein the hydrogel is formed during polymerization with monomer containing two or more ethylenic groups.
